(19) United States
(12) Patent Application Publication
(10) Pub. No.: US 2025/0065083 A1
(43) Pub. Date: Feb. 27, 2025

(54) Rapidly Insertable Central Catheters, Assemblies, and Methods Thereof

(71) Applicant: Bard Access Systems, Inc., Salt Lake City, UT (US)

(72) Inventors: Bryan Haymond, Emigration Canyon, UT (US); Glade H. Howell, Draper, UT (US)

(21) Appl. No.: 18/940,343

(22) Filed: Nov. 7, 2024

Related U.S. Application Data
(63) Continuation of application No. 17/554,978, filed on Dec. 17, 2021, now Pat. No. 12,138,405.
(60) Provisional application No. 63/126,997, filed on Dec. 17, 2020.

(51) Int. Cl. A61M 25/06; A61M 25/00; A61M 25/09

(52) U.S. Cl. CPC A61M 25/0631; A61M 25/0097; A61M 25/0606; A61M 25/0637; A61M 25/09

(57) ABSTRACT

Rapidly insertable central catheters (“RICCs”), RICC assemblies, and methods thereof. A RICC can include a catheter tube, a suture wing disposed over a medial portion of the catheter tube, a hub coupled to a proximal portion of the catheter tube, and a number of extension legs extending from the hub. The catheter tube can include a first section in a distal portion of the catheter tube and a second section proximal of the first section. The suture wing can include a projection opposite a patient-facing side of the suture wing and a needle through hole through the projection. The needle through hole can be configured to accept a needle therethrough for insertion of the needle into a primary lumen of the catheter tube, which passes through a catheter-tube through hole through the suture wing.

### CROSS-REFERENCE

This application is a continuation of U.S. patent application Ser. No. 17/554,978, filed Dec. 17, 2021, now U.S. Pat. No. 12,138,405, which claims the benefit of priority to U.S. Provisional Application No. 63/126,997, filed Dec. 17, 2020, which is incorporated by reference in its entirety into this application.

### BACKGROUND

The Seldinger technique utilizes a number of steps and medical devices (e.g., a needle, a scalpel, a guidewire, an introducer sheath, a dilator, etc.) for introducing central venous catheters (“CVCs”) and the like into patients and advancing such catheters through vasculatures of the patients. While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the number of steps of the Seldinger technique. As such, there is a need to reduce the number of steps and medical devices involved in introducing a catheter into a patient and advancing the catheter through a vasculature thereof.

Disclosed herein are rapidly insertable central catheters (“RICCs”), RICC assemblies, and methods thereof that address the foregoing.

### SUMMARY

Disclosed herein is a rapidly insertable central catheter (“RICC”) including, in some embodiments, a catheter tube, a suture wing disposed over a medial portion of the catheter tube, a hub coupled to a proximal portion of the catheter tube, and a number of extension legs extending from the hub. The catheter tube includes a first section in a distal portion of the catheter tube and a second section proximal of the first section. The suture wing includes a projection opposite a patient-facing side of the suture wing and a needle through hole through the projection. The needle through hole is configured to accept a needle therethrough for insertion of the needle into a primary lumen of the catheter tube, which passes through a catheter-tube through hole through the suture wing. The number of extension legs are equal to a number of lumens extending through the RICC.

In some embodiments, the suture wing further includes a septum disposed in the needle through hole. The septum is configured to provide a fluid-tight seal for the needle through hole during a percutaneous puncture with the needle disposed in the primary lumen of the catheter tube. The septum is also configured to provide a fluid-tight seal for the needle through hole subsequent to withdrawal of the needle from the needle through hole after the percutaneous puncture.

In some embodiments, the first section of the catheter tube is of a first polymeric material having a first durometer and the second section of the catheter tube is of at least a second polymeric material having a second durometer less than the first durometer.

In some embodiments, each polymeric material of the first and second polymeric materials is a polyurethane.

In some embodiments, the second section includes an outer layer of the catheter tube extruded over an inner layer of the catheter tube such that an outer diameter of the catheter tube is larger in the second section than the first section.

In some embodiments, the catheter tube further includes a bump demarcating a third section of the catheter tube proximal of the second section. The third section has a larger outer diameter than both the first and second sections of the catheter tube.

In some embodiments, the suture wing is disposed over the bump of the catheter tube such that the needle through hole aligns with a needle eyelet in the catheter tube and the catheter tube proximal of the suture wing has a larger outer diameter than the catheter tube distal of the suture wing.

In some embodiments, the RICC is a triluminal catheter including a trifurcated hub as the hub and three extension legs for the number of extension legs extending from the hub. Each extension leg of the three extension legs includes a Luer connector coupled to a proximal portion of the extension leg.

In some embodiments, the RICC includes the primary lumen extending from an opening in a proximal end of a first Luer connector to an opening in a distal end of the first section of the catheter tube, a secondary lumen extending from an opening in a proximal end of a second Luer connector to a first eyelet in a distal portion of the second section of the catheter tube, and a tertiary lumen extending from an opening in a proximal end of a third Luer connector to a second eyelet in the distal portion of the second section of the catheter tube proximal of the first eyelet.

Also disclosed herein is a RICC assembly including, in some embodiments, a RICC and a needle preloaded in the RICC. The RICC includes a catheter tube, a suture wing disposed over a medial portion of the catheter tube, and a hub coupled to a proximal portion of the catheter tube. The catheter tube includes a first section in a distal portion of the catheter tube and a second section proximal of the first section. The suture wing includes a projection opposite a patient-facing side of the suture wing and a needle through hole through the projection. The catheter tube passes through a catheter-tube through hole through the suture wing, and the needle is inserted into a primary lumen of the catheter tube by way of the needle through hole of the suture wing.

In some embodiments, a distal tip of the needle extends past a distal end of the catheter tube for a percutaneous puncture with the needle.

In some embodiments, the RICC assembly further includes a guidewire preloaded in the RICC. The guidewire is inserted into the primary lumen of the catheter tube proximal of an entry point of the needle in the primary lumen of the catheter tube.

In some embodiments, the suture wing further includes a septum disposed in the needle through hole. The septum is configured to provide a fluid-tight seal for the needle through hole during a percutaneous puncture with the needle disposed in the primary lumen of the catheter tube. The septum is also configured to provide a fluid-tight seal for the needle through hole subsequent to withdrawal of the needle from the needle through hole after the percutaneous puncture.

In some embodiments, the first section of the catheter tube is of a first polymeric material having a first durometer and the second section of the catheter tube is of at least a second polymeric material having a second durometer less than the first durometer.

In some embodiments, each polymeric material of the first and second polymeric materials is a polyurethane.

In some embodiments, the second section includes an outer layer of the catheter tube extruded over an inner layer of the catheter tube such that an outer diameter of the catheter tube is larger in the second section than the first section.

In some embodiments, the catheter tube further includes a bump demarcating a third section of the catheter tube proximal of the second section. The third section has a larger outer diameter than both the first and second sections of the catheter tube.

In some embodiments, the suture wing is disposed over the bump of the catheter tube such that the needle through hole aligns with a needle eyelet in the catheter tube and the catheter tube proximal of the suture wing has a larger outer diameter than the catheter tube distal of the suture wing.

In some embodiments, the RICC is a triluminal catheter including a trifurcated hub as the hub and three extension legs extending from the hub. Each extension leg of the three extension legs includes a Luer connector coupled to a proximal portion of the extension leg.

In some embodiments, the RICC includes the primary lumen extending from an opening in a proximal end of a first Luer connector to an opening in a distal end of the first section of the catheter tube, a secondary lumen extending from an opening in a proximal end of a second Luer connector to a first eyelet in a distal portion of the second section of the catheter tube, and a tertiary lumen extending from an opening in a proximal end of a third Luer connector to a second eyelet in the distal portion of the second section of the catheter tube proximal of the first eyelet.

Also disclosed herein is a method for introducing a RICC into a blood-vessel lumen of a patient. The method includes, in some embodiments, a RICC assembly-obtaining step, a needle tract-establishing step, RICC-advancing step, and a needle-withdrawing step. The RICC assembly-obtaining step includes obtaining a RICC assembly. The RICC assembly includes the RICC and a needle preloaded in the RICC in a ready-to-introduce state of the RICC assembly. The RICC includes a catheter tube, a suture wing disposed over a medial portion of the catheter tube, and a hub coupled to a proximal portion of the catheter tube. A shaft of the needle extends through a needle through hole through the suture wing and a primary lumen of the catheter tube in the ready-to-introduce state of the RICC assembly. In addition, a distal tip of the needle extends beyond a distal end of the catheter tube in the ready-to-introduce state of the RICC assembly. The needle tract-establishing step includes establishing a needle tract from an area of skin to the blood-vessel lumen with the distal tip of the needle. The RICC-advancing step includes advancing a distal portion of the catheter tube into the blood-vessel lumen over the needle. The needle-withdrawing step includes withdrawing the needle from the RICC by way of the needle through hole of the suture wing.

In some embodiments, the method further includes a syringe-connecting step and a blood-aspirating step. The syringe-connecting step is optional in that it includes connecting a syringe to the needle if the syringe is not already connected to the needle in the ready-to-introduce state of the RICC assembly. The blood-aspirating step includes aspirating blood with the syringe before the needle-withdrawing step. The blood-aspirating step confirms the distal tip of the needle is disposed in the blood-vessel lumen.

In some embodiments, the method further includes a guidewire-advancing step. The guidewire-advancing step includes advancing a guidewire into the blood-vessel lumen beyond the distal end of the catheter tube after the needle-withdrawing step. The guidewire is preloaded in the RICC proximal of an entry point of the needle in the primary lumen of the catheter tube in the ready-to-introduce state of the RICC assembly.

In some embodiments, the catheter tube includes a first section of a first polymeric material having a first durometer and a second section proximal of the first section of at least a second polymeric material having a second durometer less than the first durometer. The catheter tube is thusly configured with a column strength for advancing at least the distal portion of the catheter tube into the blood-vessel lumen without buckling.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a RICC assembly in an assembled, ready-to-introduce state in accordance with some embodiments.

FIG. 2 illustrates the RICC assembly in a disassembled state in accordance with some embodiments.

FIG. 3 illustrates a RICC in a disassembled state in accordance with some embodiments.

FIG. 4 illustrates an end view of a suture wing of the RICC in accordance with some embodiments.

FIG. 5 illustrates a side view of the suture wing over a catheter tube of the RICC in accordance with some embodiments.

FIG. 6 illustrates an alternative suture wing-and-hub combination of a RICC in accordance with some embodiments.

FIG. 7 illustrates a distal portion of the catheter tube of the RICC of FIG. 3 in accordance with some embodiments.

FIG. 8A illustrates a transverse cross section of a first section of the catheter tube in accordance with some embodiments.

FIG. 8B illustrates a transverse cross section of a transition between the first section and a second section of the catheter tube in accordance with some embodiments.

FIG. 8C illustrates another transverse cross section of the transition between the first section and the second section of the catheter tube in accordance with some embodiments.

FIG. 8D illustrates a transverse cross section of the second section of the catheter tube in accordance with some embodiments.

FIG. 9 illustrates a portion of the manufacturing of the catheter tube in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, there is a need to reduce the number of steps and medical devices involved in introducing a catheter into a patient and advancing the catheter through a vasculature thereof. Disclosed herein are RICCs, RICC assemblies, and methods thereof that address the foregoing.

### Rapidly Insertable Central Catheters

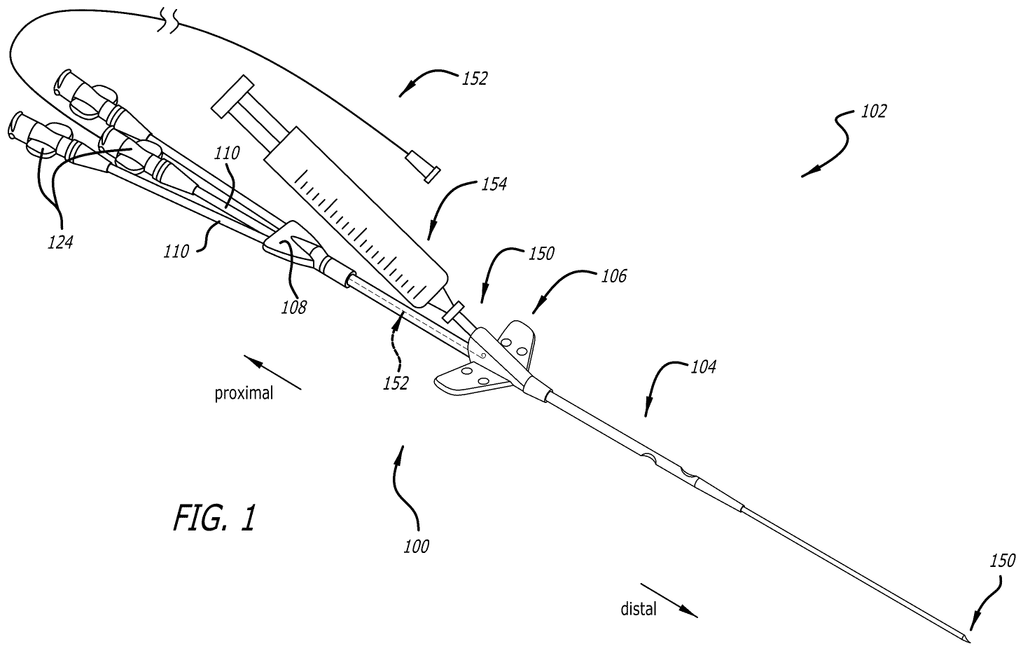

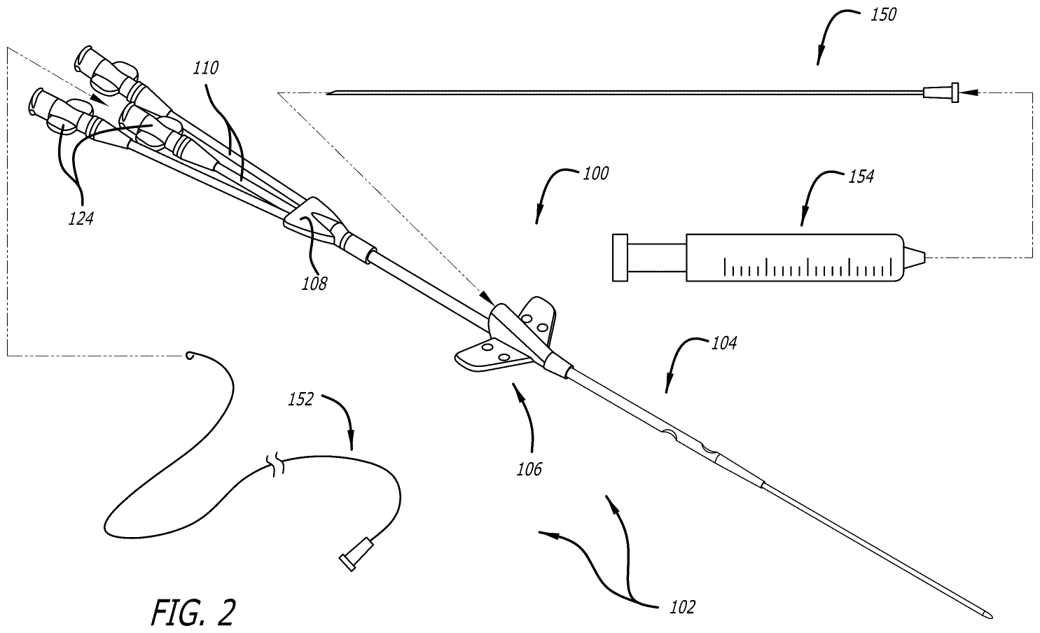

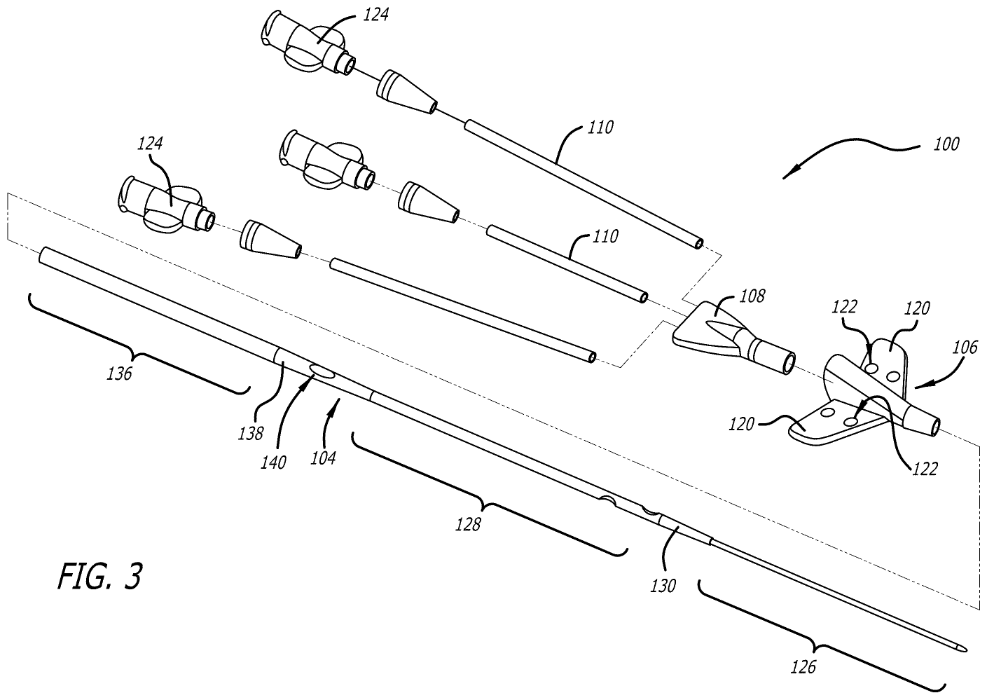

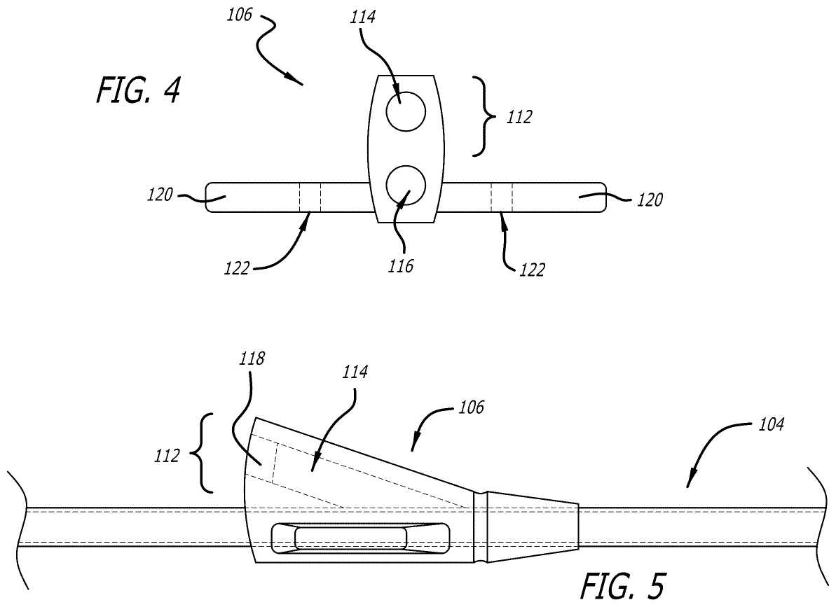

FIGS. 1 and 2 illustrate a RICC 100 in a RICC assembly 102 in accordance with some embodiments. FIG. 3 illustrates the RICC 100 in a disassembled state in accordance with some embodiments.

As shown, the RICC 100 includes a catheter tube 104, a suture wing 106, a hub 108, and a number of extension legs 110 extending from the hub 108.

The suture wing 106 is disposed over a medial portion of the catheter tube 104 between a proximal portion and a distal portion of the catheter tube 104. The suture wing 106 includes a projection 112 opposite a patient-facing side of the suture wing 106 and a needle through hole 114 through the projection 112. The needle through hole 114 is configured to accept a needle (e.g., the needle 150) therethrough for insertion of the needle into a primary lumen of the RICC 100, specifically the primary catheter-tube lumen 146 of the catheter tube 104, which catheter tube 104 passes through a catheter-tube through hole 116 through the suture wing 106.

The suture wing 106 further includes a septum 118 disposed in the needle through hole 114. The septum 118 is configured to provide a fluid-tight seal for the needle through hole 114 during a percutaneous puncture with the needle (e.g., the needle 150) when disposed in the primary lumen of the RICC 100 (e.g., the primary catheter-tube lumen 146 of the catheter tube 104) such as in the ready-to-introduce state of the RICC assembly 102 set forth below. The septum 118 is also configured to provide a fluid-tight seal for the needle through hole 114 subsequent to withdrawal of the needle from the needle through hole 114 after the percutaneous puncture.

The suture wing 106 includes a pair of wings 120 including a number of wing through holes 122 for suturing the suture wing 106 to a patient. Each wing of the pair of wings 120 can include one wing through hole, two wing through holes, three wing through holes, or four wing through holes for suturing the suture wing 106 to a patient.

FIG. 6 illustrates an alternative suture wing-and-hub combination 107 of the RICC 100 in accordance with some embodiments.

As shown, the suture wing-and-hub combination 107, like the suture wing 106, includes the needle through hole 114 configured to accept a needle (e.g., the needle 150) therethrough for insertion of the needle into the primary lumen of the RICC 100, specifically the primary catheter-tube lumen 146 of the catheter tube 104, which catheter tube 104 passes through the catheter-tube through hole 116 through the suture wing 106.

Each of the suture wing 106 and the suture wing-and-hub combination 107 provides an advantage in using the RICC 100 over any other RICC currently in development in that the needle used for establishing a percutaneous puncture with the RICC 100 can be shorter than that needed for the other RICC. Indeed, the other RICC requires a relatively long needle disposed in a primary lumen thereof that extends from an opening in a proximal end of the other RICC (e.g., an opening of a Luer connector) to an opening in a distal end thereof. With such a long needle, it can take a relatively long time to witness a flashback of blood after a percutaneous puncture with the needle. In addition, such a long needle is prone to intraluminal clots. With the RICC 100, however, the needle can be shorter than that needed for the other RICC, thereby providing a relatively short time to witness a flashback of blood after a percutaneous puncture with the needle as well as reduced risk of intraluminal clots in the needle.

The hub 108 is coupled to the proximal portion of the catheter tube 104 such as by insertion of the proximal portion of the catheter tube 104 into a bore in a distal portion of the hub 108. While not shown, the hub 108 also includes a number of bores in a proximal portion of the hub 108 corresponding in number to the number of extension legs 110. The number of bores in the distal portion of the hub 108 are configured to accept insertion of the number of extension legs 110 into the number of bores.

The RICC 100 can be a monoluminal catheter or a multiluminal catheter such as a diluminal catheter, a triluminal catheter, a tetraluminal catheter, a pentaluminal catheter, or a hexaluminal catheter. Accordingly, the hub 108 is either not furcated in accordance with the monoluminal catheter or furcated in accordance with a number of lumens extending through the RICC 100. For example, the hub 108 can be bifurcated for the diluminal catheter or trifurcated for the triluminal catheter. Depending upon a chosen method of manufacturing, the hub 108 can be molded over a number of core pins for a number of fluid pathways longitudinally extending through the hub 108 configured to fluidly connect a number of catheter-tube lumens of the catheter tube 104 to a number of extension-leg lumens of the number of extension legs 110. Alternatively, the hub 108 can be molded over a number of cannulas longitudinally extending through the hub 108 configured to fluidly connect the number of catheter-tube lumens of the catheter tube 104 to the number of extension-leg lumens of the number of extension legs 110.

The number of extension legs 110 extend from the hub 108 by way of their distal portions. The number of extension legs 110 is equal to the number of lumens extending through the RICC 100. For example: If the RICC 100 is a monoluminal catheter, one extension leg extends from the hub 108. If the RICC 100 is a diluminal catheter, two extension legs extend from the hub 108. If the RICC 100 is a triluminal catheter, three extension legs extend from the hub 108.

The RICC 100 further includes a number of Luer connectors 124 for fluidly connecting a number of medical devices to the RICC 100. Each extension leg of the number of extension legs 110 includes a Luer connector of the number of Luer connectors 124 coupled to a proximal portion of the extension leg. Given the foregoing, the number of Luer connectors 124 is equal to the number of extension legs 110, which number of extension legs 110, in turn, is equal to the number of lumens extending through the RICC 100. For example: If the RICC 100 is a monoluminal catheter, one extension leg extends from the hub 108 and one Luer connector is coupled to the one extension leg. If the RICC 100 is a diluminal catheter, two extension legs extend from the hub 108 and two Luer connectors are respectively coupled to the two extension legs. If the RICC 100 is a triluminal catheter, three extension legs extend from the hub 108 and three Luer connectors are respectively coupled to the three extension legs.

The catheter tube 104 includes at least a first section 126 in the distal portion of the catheter tube 104 and a second section 128 proximal of the first section 126 of the catheter tube 104. The catheter tube 104 can include a transition 130 between the first section 126 and the second section 128 of the catheter tube 104 in accordance with the method of manufacturing the catheter tube 104 set forth below. Indeed, in accordance with the manufacturing method set forth below, the transition 130 and the second section 128 of the catheter tube 104 include an outer layer 132 (see FIGS. 8B-8D) of the catheter tube 104 extruded over an inner layer 134 (see FIGS. 8A-8D) of the catheter tube 104 such that an outer diameter of the catheter tube 104 is larger in the second section 128 than the first section 126 of the catheter tube 104 commencing with the transition 130 between the first section 126 and the second section 128 of the catheter tube 104.

The first section 126 of the catheter tube 104 as well as the inner layer 134 of both the transition 130 and the second section 128 of the catheter tube 104 can be formed of a first polymeric material (e.g., polytetrafluoroethylene, polypropylene, or a polyurethane) having a first durometer, while a remainder of the transition 130 and the second section 128 of the catheter tube 104, namely the outer layer 132 thereof, can be formed of a second polymeric material (e.g., polyvinyl chloride, polyethylene, a polyurethane, or silicone) having a second durometer less than the first durometer, more than the first durometer, or substantially equal to the first durometer. For example, each layer of the inner layer 134 and the outer layer 132 of the catheter tube 104 can be made from a different polyurethane (e.g., a same or different diisocyanate or triisocyanate reacted with a different diol or triol, a different diisocyanate or triisocyanate reacted with a same or different diol or triol, etc.) having a different durometer. Polyurethane is advantageous for the catheter tube 104 in that polyurethane can be relatively rigid at room-temperature but become more flexible in vivo at body temperature, which reduces irritation to vessel walls and phlebitis. Polyurethane is also advantageous in that can be less thrombogenic than some other polymers.

Notwithstanding the foregoing, the first section 126 and the second section of the catheter tube 104, which include both the inner layer 134 and the outer layer 132 of the catheter tube 104, can be formed of a same polymeric material (e.g., a polyurethane) with a same durometer provided a column strength of the catheter tube 104 is sufficient to prevent buckling of the catheter tube 104 when inserted into an insertion site and advanced through a vasculature of a patient. The column strength of the catheter tube 104 in any given embodiment is notable in that the column strength makes it possible to rapidly insert the catheter tube 104 into an insertion site and advance the catheter tube 104 through a vasculature of a patient without the using the Seldinger technique.

It should be understood the first durometer and the second durometer can be on different scales (e.g., Type A or Type D). Thus, even if the second durometer of the second polymeric material is less than the first durometer of the first polymeric material, the second durometer might not be numerically less than the first durometer. Likewise, even if the second durometer of the second polymeric material is more than the first durometer of the first polymeric material, the second durometer might not be numerically more than the first durometer. That said, the hardness of the second polymeric material can still be respectively less or more than the hardness of the first polymeric material as the different scales—each of which ranges from 0 to 100—are designed for characterizing different materials in groups of the materials having a like hardness.

The catheter tube 104 can include a third section 136 proximal of the second section 128 of catheter tube 104 including a bumped diameter demarcated by a bump 138 in the medial portion of the catheter tube 104. The third section 136 of the catheter tube 104 has a larger outer diameter than both the first section 126 and the second section 128 of the catheter tube 104. The suture wing 106 can be disposed over the bump 138 as shown among FIGS. 1 and 2 such that the needle through hole 114 aligns with an optional needle eyelet 140 in the catheter tube 104. Thus, the catheter tube 104 proximal of the suture wing 106 (also known as a catheter-tube extension herein) has a larger outer diameter than the catheter tube 104 distal of the suture wing 106. The larger outer diameter of the third section 136 of the catheter tube 104 proximal of the suture wing 106 provides a thicker, more kink-resistant catheter-tube wall useful for bending the hub 108 and the number of extension legs 110 away from a head or neck of a patient while the RICC 100 is in use. In addition, any lumens present in the catheter tube 104 can have a greater diameter in the third section 136 of catheter tube 104 proximal of the suture wing 106 than distal of the suture wing 106. This prevents flow rate reduction, particularly when the third section 136 of the catheter tube 104 proximal of the suture wing 106 is bent away from a head or neck of a patient.

The catheter tube 104 between the suture wing 106 and the hub 108 can have a reverse taper in which the larger outer diameter of the catheter tube 104 continues to increase from the suture wing 106 to the hub 108. In other words, the catheter tube 104 tapers from the hub 108 to the suture wing 106 but continues to have a larger outer diameter than the catheter tube 104 distal of the suture wing 106. In association with the continuously increasing outer diameter of the catheter tube 104 from the suture wing 106 to the hub 108, the catheter-tube wall can continuously increase in thickness, any lumens of the catheter tube 104 can continuously increase in cross-sectional area, or a combination thereof. Consequently, the catheter tube 104 between the suture wing 106 and the hub 108 can be more resistant to kinks and flow rate reduction, particularly when the catheter tube 104 proximal of the suture wing 106 is bent away from a head or neck of a patient. Notwithstanding the foregoing, the catheter tube 104 between the suture wing 106 and the hub 108 can alternatively have a constant diameter from the suture wing 106 to the hub 108.

Advantageously, the catheter tube 104 between the suture wing 106 and the hub 108, namely the third section 136 of the catheter tube 104 or the catheter-tube extension, is a single catheter tube configured to abate bacterial ingress between a dressing applied over the suture wing 106 and skin of a patient. Existing CVCs or peripherally inserted central catheters (“PICCs”) have multiple extension legs extending from suture wing-hub combinations common to the CVCs and PICCs. The multiple extension legs in the CVCs or PICCs provide multiple pathways under the dressing for microbial ingress. The catheter tube 104 being a single catheter tube between at least the suture wing 106 and the hub 108 enables the dressing to be pinched more tightly around the catheter tube 104 than possible for the multiple extension legs of the existing CVCs or PICCs. For example, the dressing can be easily wrapped around an entirety of the catheter tube 104 and pinched together under the catheter tube 104 between the catheter tube 104 and the patient. In contrast, even wrapping the dressing around the multiple extension legs of the existing CVCs or PICCs as described for the catheter-tube extension leaves gaps between adjacent extension tubes for bacterial ingress. Thus, the catheter tube 104 being a single catheter tube limits bacterial ingress between the dressing applied over the suture wing 106 and the skin of the patient.

The catheter tube 104 between the suture wing 106 and the hub 108, again the third section 136 of the catheter tube 104 or the catheter-tube extension, is also configured to mitigate patient discomfort from proximity of the number or extension legs 110 to a head or neck of the patient. As set forth above, the third section 136 of the catheter tube 104 proximal of the suture wing 106 provides a thicker, more kink-resistant catheter-tube wall; however, the third section 136 of the catheter tube 104 is flexible enough to enable the catheter tube 104 to be bent away from the head or neck of the patient and secured to the patient for his or her comfort.

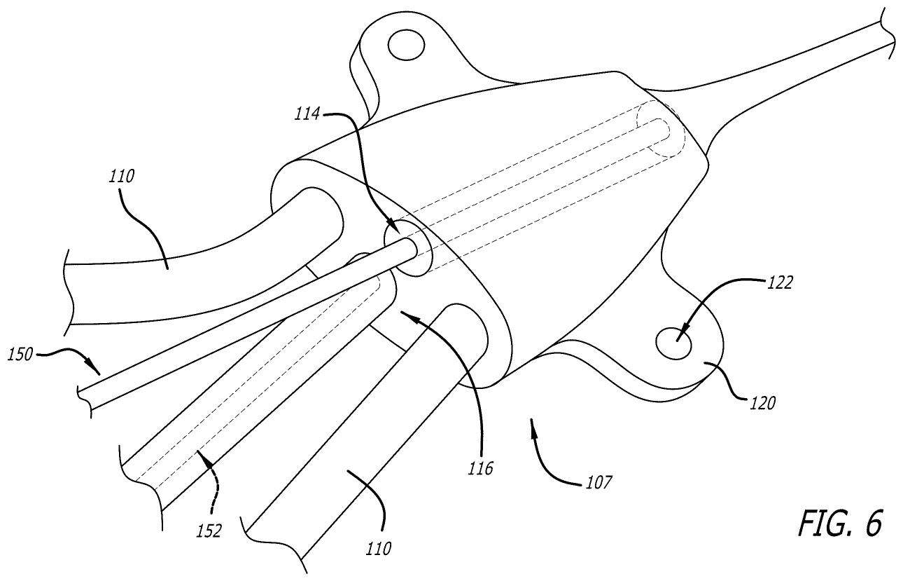

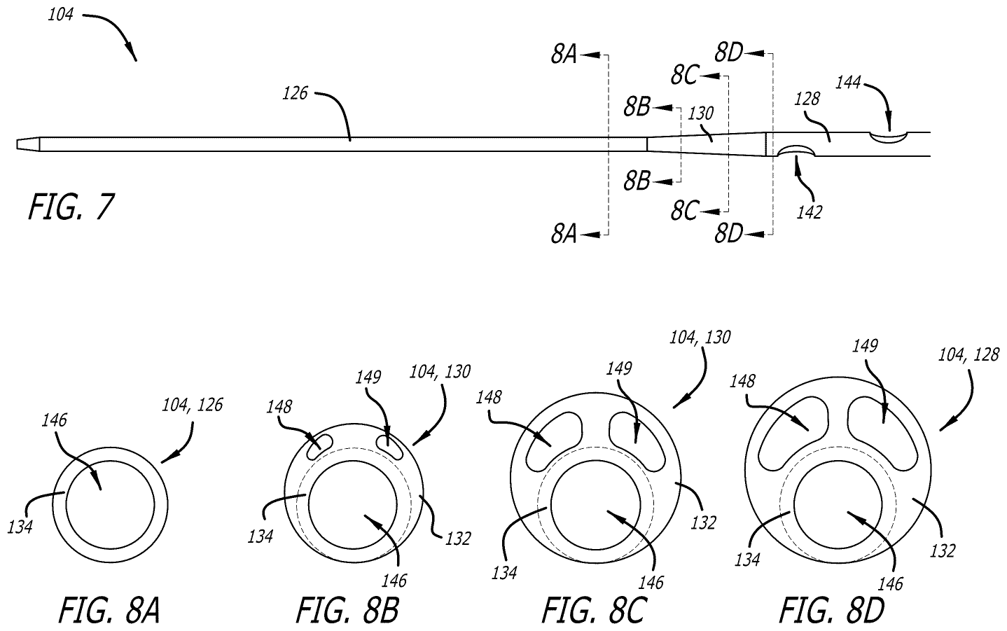

FIG. 7 illustrates a distal portion of the catheter tube 104 of the RICC 100 in accordance with some embodiments. FIGS. 8A-8D illustrate various transverse cross sections of the catheter tube 104 in accordance with some embodiments.

Again, the RICC 100 can be a monoluminal catheter or a multiluminal catheter such as a diluminal catheter, a triluminal catheter, a tetraluminal catheter, a pentaluminal catheter, or a hexaluminal catheter. The catheter tube 104 can correspondingly be a monoluminal catheter tube or a multiluminal catheter tube such as a diluminal catheter tube, a triluminal catheter tube, a tetraluminal catheter tube, a pentaluminal catheter tube, or a hexaluminal catheter tube.

When the RICC 100 is configured as a triluminal catheter as shown among FIGS. 1-3, 7, and 8A-8D, the RICC 100 includes a primary lumen, a secondary lumen, and a tertiary lumen. The primary lumen extends from an opening in a proximal end of a first Luer connector of the number of Luer connectors 124 to an opening in a tip or distal end of the first section 126 of the catheter tube 104. The secondary lumen extends from an opening in a proximal end of a second Luer connector of the number of Luer connectors 124 to an eyelet 142 in a distal portion of the second section 128 of the catheter tube 104. The tertiary lumen extends from an opening in a proximal end of a third Luer connector of the number of Luer connectors 124 to an eyelet 144 proximal of the eyelet 142 in the distal portion of the second section 128 of the catheter tube 104. Each lumen of the primary lumen, the secondary lumen, and the tertiary lumen is further described in a separate paragraph set forth below.

The primary lumen of the RICC 100 includes fluidly connected luminal sections including a primary catheter-tube lumen 146 extending along an entire length of the catheter tube 104, a primary fluid passageway or primary cannula lumen of the hub 108, a primary extension-leg lumen of a first extension leg of the number of extension legs 110, and a primary Luer-connector lumen of the first Luer connector of the number of Luer connectors 124.

The secondary lumen of the RICC 100 includes fluidly connected luminal sections including a secondary catheter-tube lumen 148, which proximally extends from the eyelet 142 in the distal portion of the second section 128 of the catheter tube 104 along a remainder of the catheter tube 104. The fluidly connected luminal sections of the secondary lumen of the RICC 100 further include a secondary fluid passageway or secondary cannula lumen of the hub 108, a secondary extension-leg lumen of a second extension leg of the number of extension legs 110, and a secondary Luer-connector lumen of the second Luer connector of the number of Luer connectors 124.

The tertiary lumen of the RICC 100 includes fluidly connected luminal sections including a tertiary catheter-tube lumen 149, which proximally extends from the eyelet 144 in the distal portion of the second section 128 of the catheter tube 104 along a remainder of the catheter tube 104. The fluidly connected luminal sections of the tertiary lumen of the RICC 100 further include a tertiary fluid passageway or tertiary cannula lumen of the hub 108, a tertiary extension-leg lumen of a third extension leg of the number of extension legs 110, and a tertiary Luer-connector lumen of the third Luer connector of the number of Luer connectors 124.

When the RICC 100 is configured as a diluminal catheter, the RICC 100 includes a primary lumen and a secondary lumen. Like the RICC 100 when configured as the triluminal catheter, the primary lumen extends from the opening in the proximal end of the first Luer connector of the number of Luer connectors 124 to the opening in the tip or the distal end of the first section 126 of the catheter tube 104. The secondary lumen extends from the opening in the proximal end of the second Luer connector of the number of Luer connectors 124 to the eyelet 142 in the distal portion of the second section 128 of the catheter tube 104. Because the primary lumen and the secondary lumen of the RICC 100 configured as the diluminal catheter are analogous to the primary lumen and the secondary lumen of the RICC 100 configured as the triluminal catheter, additional detail for each lumen of the primary lumen and the secondary lumen of the RICC 100 configured as the diluminal catheter can be discerned from the description set forth above for the primary lumen and the secondary lumen of the RICC 100 configured as the triluminal catheter.

When the RICC 100 is configured as a monoluminal catheter, the RICC 100 includes a single lumen, which single lumen is also known as a primary lumen herein for consistency with description set forth above. Like the RICC 100 when configured as the triluminal catheter, the primary lumen extends from the opening in the proximal end of the first Luer connector of the number of Luer connectors 124 to the opening in the tip or the distal end of the first section 126 of the catheter tube 104. Because the primary lumen of the RICC 100 configured as the monoluminal catheter is analogous to the primary lumen of the RICC 100 configured as the triluminal catheter, additional detail for the primary lumen of the RICC 100 configured as the monoluminal catheter can be discerned from the description set forth above for the primary lumen of the RICC 100 configured as the triluminal catheter.

### RICC Assemblies

FIGS. 1 and 2 illustrate the RICC assembly 102 in accordance with some embodiments. In particular, FIG. 1 illustrates the RICC assembly 102 in an assembled, ready-to-introduce state in accordance with some embodiments, whereas FIG. 2 illustrates the RICC assembly 102 in a disassembled state in accordance with some embodiments.

As shown, the RICC assembly 102 includes at least the RICC 100 and a needle 150 preloaded in the RICC 100 in the ready-to-introduce state of the RICC assembly 102. Optionally, the RICC assembly 102 includes a guidewire 152 preloaded in the RICC 100, a syringe 154 coupled to a hub of the needle 150, or both the guidewire 152 and the syringe 154 in the ready-to-introduce state of the RICC assembly 102. In a packaged state of the RICC assembly 102, the RICC assembly 102 resembles the ready-to-introduce state of the RICC assembly 102; however, if the syringe 154 is present in a package including the RICC assembly 102, the syringe 154 need not be coupled to the hub of the needle 150. Indeed, the syringe 154 can be packaged alongside a remainder of the RICC assembly 102 in the packaged state of the RICC assembly 102.

The needle 150 is inserted into the primary lumen of the RICC 100 by way of the needle through hole 114 of the suture wing 106 in the ready-to-introduce state of the RICC assembly 102. Specifically, the needle 150 is inserted into the primary catheter-tube lumen 146 of the catheter tube 104, which catheter tube 104 passes through the catheter-tube through hole 116 through the suture wing 106. In addition, a distal tip of the needle 150 including a bevel extends past the distal end of the catheter tube 104 for a percutaneous puncture with the needle 150.

The guidewire 152 is inserted into the primary lumen of the RICC 100 by way of the opening in the proximal end of the first Luer connector of the number of Luer connectors 124 in the ready-to-introduce state of the RICC assembly 102. A distal end of the guidewire 152 is proximal of an entry point of the needle 150 in the primary lumen of the RICC 100, specifically the primary catheter-tube lumen 146 of the catheter tube 104. Such placement of the guidewire 152 in the ready-to-introduce state of the RICC assembly 102 enables the guidewire 152 to be immediately advanced into a blood-vessel lumen of a patient after a percutaneous puncture with the needle 150 and withdrawal thereof from the RICC 100. The foregoing placement of the guidewire 152 in the ready-to-introduce state of the RICC assembly 102 is advantageous over placement of the guidewire 152 in the needle 150 because it allows the guidewire 152 to have a larger diameter than that allowed by the needle 150, which larger diameter provides more stability for the catheter tube 104 when maneuvered over the guidewire 152.

### Methods

Methods of the RICCs and RICC assemblies set forth above include methods of making and using the RICCs and RICC assemblies. An example method for making the RICC 100 is set forth below followed by a method for using the RICC assembly 102, specifically a method for introducing the RICC 100 into a blood-vessel lumen of a patient.

A method for making the RICC 100 includes one or more catheter tube-manufacturing steps for manufacturing the catheter tube 104, one or more extruding steps of extruding one or more extrudable components other than the catheter tube 104 such as the number of extension legs 110, one or more molding steps of molding one or more moldable components, and one or more assembling steps of assembling the RICC 100 or any portion thereof by coupling the extrudable components including the catheter tube 104 and the moldable components together.

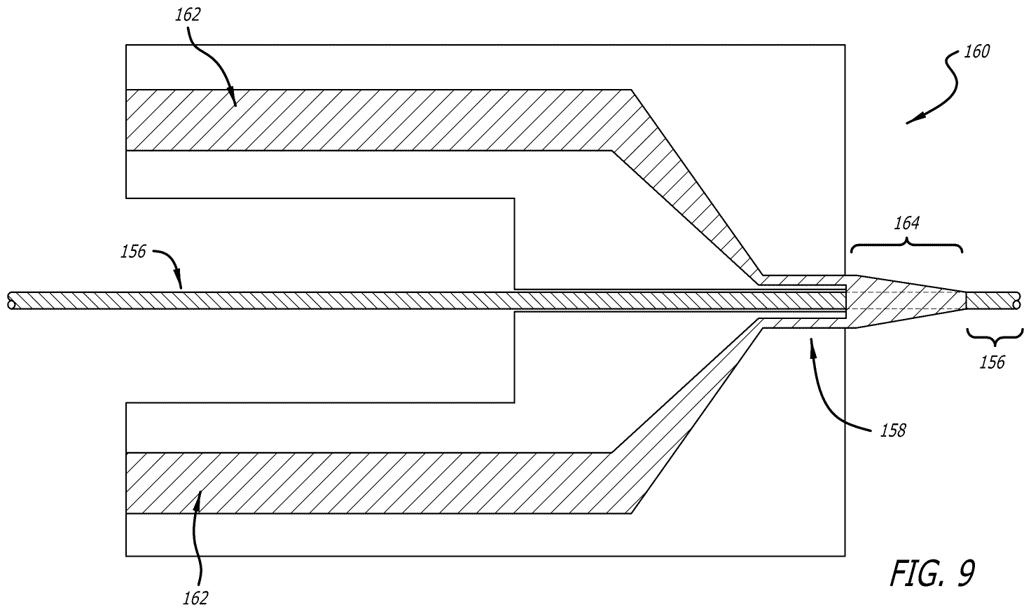

FIG. 9 illustrates a portion of the manufacturing of the catheter tube 104 in accordance with some embodiments.

The one-or-more catheter tube-manufacturing steps include an inner-layer forming step. The inner-layer forming step includes forming the inner layer 134 of the catheter tube 104 by extruding monoluminal tubing 156 of the first polymeric material.

The one-or-more catheter tube-manufacturing steps further include an inserting step. The inserting step includes inserting an end of the monoluminal tubing 156 through a die 158 of an extruder 160.

The one-or-more catheter tube-manufacturing steps further include a second-layer forming step. The second-layer forming step includes forming the outer layer 132 of the catheter tube 104 by periodically forcing a melt 162 of the second polymeric material through the die 158 around the monoluminal tubing 156, thereby forming output tubing with sections of mixed-layer tubing 164 regularly interspersed with sections of the monoluminal tubing 156.

The one-or-more catheter tube-manufacturing steps can further include a bonding layer-applying step. The bonding layer-applying step includes applying a bonding layer over the monoluminal tubing 156 before forcing the melt 162 of the second polymeric material through the die 158 around the monoluminal tubing 156 in the second-layer forming step.

The one-or-more catheter tube-manufacturing steps further includes a lumen forming step. The lumen forming step includes forming one or more additional lumens (e.g., the secondary catheter-tube lumen 148, the tertiary catheter-tube lumen 149, etc.) to that of the monoluminal tubing 156 by injecting air into the melt 162 of the second polymeric material while forcing the melt 162 of the second polymeric material through the die 158 around the monoluminal tubing 156.

The one-or-more catheter tube-manufacturing steps further includes an eyelet-creating step. The eyelet-creating step includes creating one or more eyelets (e.g., the needle eyelet 140, the eyelet 142, the eyelet 144, etc.) in the sections of the mixed-layer tubing 164 to correspondingly establish one or openings to the primary catheter-tube lumen 146 or the one-or-more additional lumens.

The one-or-more catheter tube-manufacturing steps can further include a bump-forming step. The bump-forming step includes forming bumps (e.g., the bump 138 in the medial portion of the catheter tube 104) in the sections of the mixed-layer tubing 164 by periodically slowing a rate of pulling the output tubing with a puller to increase an outer diameter of the output tubing after the bumps.

The one-or-more catheter tube-manufacturing steps can further include a reverse-tapering step. The reverse-tapering step includes reverse tapering the outer diameter in the sections of the mixed-layer tubing 164 after the bumps by continuously slowing the rate of pulling the output tubing with the puller.

The one-or-more catheter tube-manufacturing steps can further include a cooling step. The cooling step includes pulling the output tubing through a cooling bath with the puller to cool the output tubing.

The one-or-more catheter tube-manufacturing steps further includes a cutting step. The cutting step includes cutting the output tubing in at least the sections of the monoluminal tubing 156 with a cutter to form the catheter tubes such as the catheter tube 104.

The one-or-more extruding steps can include extruding any one or more extension legs of the number of extension legs 110 in accordance with description set forth above for the one-or-more extension legs.

The one-or-more molding steps can include molding any one or more moldable components selected from the suture wing 106 and the hub 108 in accordance with description set forth above for such moldable components. The one-or-more molding steps can further include molding any one or more Luer connectors of the number of Luer connectors 124 in accordance with description set forth above for the one-or-more Luer connectors.

The one-or-more assembling steps of assembling the RICC 100 or any portion thereof can include assembling the RICC 100 in accordance with that shown in FIG. 3 with the understanding a distal portion of the catheter tube 104 is inserted into a proximal portion the suture wing 106.

A method for introducing the RICC 100 into a blood-vessel lumen of a patient includes a RICC assembly-obtaining step, a needle tract-establishing step, first and second RICC-advancing steps, and a needle-withdrawing step.

The RICC assembly-obtaining step includes obtaining the RICC assembly 102. As set forth above, the RICC assembly 102 includes at least the RICC 100 and the needle 150 preloaded in the RICC in the ready-to-introduce state of the RICC assembly 102. Indeed, the shaft of the needle 150 extends through the needle through hole 114 of the suture wing 106 and the primary lumen of the RICC 100, specifically the primary catheter-tube lumen 146 of the catheter tube 104, in the ready-to-introduce state of the RICC assembly 102. In addition, the distal tip of the needle 150 extends beyond the distal end of the catheter tube 104 in the ready-to-introduce state of the RICC assembly 102.

The needle tract-establishing step includes establishing a needle tract from an area of skin to the blood-vessel lumen with the distal tip of the needle 150.

The first RICC-advancing step includes advancing a distal portion of the catheter tube 104 into the blood-vessel lumen over the needle 150.

The needle-withdrawing step includes withdrawing the needle 150 from the RICC 100 by way of the needle through hole 114 of the suture wing 106.

The second RICC-advancing step includes advancing the catheter tube 104 through the vasculature of the patient without having to use the Seldinger technique. For example, if an insertion site is at the right subclavian vein or the right internal jugular vein, the second RICC-advancing step can include inserting the catheter tube 104 farther into the insertion site such that the catheter tube 104 or at least the distal portion thereof is advanced through the right subclavian vein or the right internal jugular vein, a right brachiocephalic vein, and into a superior vena cava. Other insertions sites such as at the left subclavian vein or the left internal jugular vein require advancing the distal portion of the catheter tube 104 through corresponding vasculature. The Seldinger technique need not be used due to the catheter tube 104 having a column strength sufficient to prevent buckling of the catheter tube 104 when inserted into the insertion site and advanced through the vasculature of the patient.

The method can further include a syringe-connecting step and a blood-aspirating step. The syringe-connecting step is optional in that it includes connecting the syringe 154 to the hub of the needle 150 if the syringe 154 is not already connected to the needle 150 in the ready-to-introduce state of the RICC assembly 102. The blood-aspirating step includes aspirating blood with the syringe 154 before the needle-withdrawing step. The blood-aspirating step confirms the distal tip of the needle 150 is disposed in the blood-vessel lumen of the patient.

The method can further include a guidewire-advancing step after the needle-withdrawing step. The guidewire-advancing step includes advancing the guidewire 152 into the blood-vessel lumen of the patient beyond the distal end of the catheter tube 104 after the needle-withdrawing step. As set forth above, the guidewire 152 is preloaded in the RICC 100 proximal of an entry point of the needle 150 in the primary lumen of the RICC 100, specifically the primary catheter-tube lumen 146 of the catheter tube 104, in the ready-to-introduce state of the RICC assembly 102.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A rapidly insertable central catheter (“RICC”), comprising:
a catheter tube including a primary lumen;
a suture wing disposed over a medial portion of the catheter tube and a needle eyelet thereof, the suture wing including:
a projection opposite a patient-facing side of the suture wing; and
a needle through hole through the projection configured to accept a needle therethrough for insertion of the needle through the needle eyelet of the catheter tube and into a primary lumen of the catheter tube, the catheter tube passing through a catheter-tube through hole through the suture wing; and
a hub coupled to a proximal portion of the catheter tube.

2. The RICC of claim 1, wherein the suture wing further includes a septum disposed in the needle through hole configured to provide a fluid-tight seal for the needle through hole during a percutaneous puncture with the needle disposed in the primary lumen of the catheter tube or subsequent to withdrawal of the needle from the needle through hole after the percutaneous puncture.

3. The RICC of claim 1, wherein the RICC is a triluminal catheter including a trifurcated hub as the hub and three extension legs extending from the hub, each extension leg of the three extension legs including a Luer connector coupled to a proximal portion of the extension leg.

4. The RICC of claim 3, wherein the RICC includes the primary lumen extending from an opening in a proximal end of a first Luer connector to an opening in a distal end of a first section of the catheter tube, a secondary lumen extending from an opening in a proximal end of a second Luer connector to a first eyelet in a distal portion of a second section of the catheter tube, and a tertiary lumen extending from an opening in a proximal end of a third Luer connector to a second eyelet in the distal portion of the second section of the catheter tube proximal of the first eyelet.

5. The RICC of claim 4, wherein the first section of the catheter tube is of a first polymeric material having a first durometer and at least a portion of the second section of the catheter tube is of a second polymeric material having a second durometer less than the first durometer.

6. The RICC of claim 4, wherein the second section includes an outer layer of the catheter tube extruded over an inner layer of the catheter tube such that an outer diameter of the catheter tube is larger in the second section than the first section.

7. The RICC of claim 4, wherein the catheter tube further includes a bump demarcating a third section of the catheter tube proximal of the second section, the third section having a larger outer diameter than both the first and second sections of the catheter tube.

8. The RICC of claim 7, wherein the suture wing is disposed over the bump of the catheter tube such that the needle through hole aligns with the needle eyelet in the catheter tube and the catheter tube proximal of the suture wing has a larger outer diameter than the catheter tube distal of the suture wing.

9. A rapidly insertable central catheter (“RICC”) assembly, comprising:
a RICC including:
a catheter tube including a primary lumen;
a suture wing disposed over a medial portion of the catheter tube and a needle eyelet thereof, the suture wing including:
a projection opposite a patient-facing side of the suture wing; and
a needle through hole through the projection, the catheter tube passing through a catheter-tube through hole through the suture wing; and
a hub coupled to a proximal portion of the catheter tube; and
a needle preloaded in the RICC, the needle inserted into a primary lumen of the catheter tube by way of the needle eyelet of the catheter tube and the needle through hole of the suture wing.

10. The RICC assembly of claim 9, wherein a distal tip of the needle extends past a distal end of the catheter tube for a percutaneous puncture with the needle.

11. The RICC assembly of claim 9, further comprising a guidewire preloaded in the RICC, the guidewire inserted into the primary lumen of the catheter tube proximal of an entry point of the needle in the primary lumen of the catheter tube.

12. The RICC assembly of claim 9, wherein the suture wing further includes a septum disposed in the needle through hole configured to provide a fluid-tight seal for the needle through hole during a percutaneous puncture with the needle disposed in the primary lumen of the catheter tube or subsequent to withdrawal of the needle from the needle through hole after the percutaneous puncture.

13. The RICC assembly of claim 9, wherein the RICC is a triluminal catheter including a trifurcated hub as the hub and three extension legs extending from the hub, each extension leg of the three extension legs including a Luer connector coupled to a proximal portion of the extension leg.

14. The RICC assembly of claim 13, wherein the RICC includes the primary lumen extending from an opening in a proximal end of a first Luer connector to an opening in a distal end of a first section of the catheter tube, a secondary lumen extending from an opening in a proximal end of a second Luer connector to a first eyelet in a distal portion of a second section of the catheter tube, and a tertiary lumen extending from an opening in a proximal end of a third Luer connector to a second eyelet in the distal portion of the second section of the catheter tube proximal of the first eyelet.

15. The RICC assembly of claim 14, wherein the first section of the catheter tube is of a first polymeric material having a first durometer and at least a portion of the second section of the catheter tube is of a second polymeric material having a second durometer less than the first durometer.

16. The RICC assembly of claim 14, wherein the second section includes an outer layer of the catheter tube extruded over an inner layer of the catheter tube such that an outer diameter of the catheter tube is larger in the second section than the first section.

17. The RICC assembly of claim 14, wherein the catheter tube further includes a bump demarcating a third section of the catheter tube proximal of the second section, the third section having a larger outer diameter than both the first and second sections of the catheter tube.

18. The RICC assembly of claim 17, wherein the suture wing is disposed over the bump of the catheter tube such that the needle through hole aligns with the needle eyelet in the catheter tube and the catheter tube proximal of the suture wing has a larger outer diameter than the catheter tube distal of the suture wing.

19. A method of manufacturing a rapidly insertable central catheter (“RICC”), comprising:
extruding a catheter tube including a primary lumen;
creating a needle eyelet in the catheter tube;
disposing a suture wing over a medial portion of the catheter tube and the needle eyelet thereof, the suture wing including:
a projection opposite a patient-facing side of the suture wing; and
a needle through hole through the projection configured to accept a needle therethrough for insertion of the needle through the needle eyelet of the catheter tube and into a primary lumen of the catheter tube, the catheter tube passing through a catheter-tube through hole through the suture wing;
coupling a hub to a proximal portion of the catheter tube.

20. The method of claim 19, wherein extruding the catheter tube includes forming a bump of larger diameter in the medial portion of the catheter tube while extruding the catheter tube, and disposing the suture wing over the medial portion of the catheter tube includes disposing the suture wing over the bump.
